(19) United States
(12) Patent Application Publication
(10) Pub. No.: US 2019/0358431 A1
(43) Pub. Date: Nov. 28, 2019

(54) CATHETER TUBE FOR MEDICAL USE

(71) Applicant: OLYMPUS CORPORATION, Tokyo (JP)

(72) Inventors: Yoshiki HATA, Tokyo (JP); Ichiro HORIUCHI, Ina-shi (JP); Kohei OGUNI, Tokyo (JP)

(73) Assignee: OLYMPUS CORPORATION, Tokyo (JP)

(21) Appl. No.: 16/538,501

(22) Filed: Aug. 12, 2019

Related U.S. Application Data
(63) Continuation of application No. PCT/JP2018/004207, filed on Feb. 7, 2018.

(30) Foreign Application Priority Data
Feb. 15, 2017 (JP) 2017-025981

(51) Int. Cl. A61M 25/00; A61M 25/09

(52) U.S. Cl. CPC A61M 25/0028; A61M 25/09; A61M 25/003

(57) ABSTRACT

A catheter tube has a single-lumen distal-side tube and a single-lumen proximal-side tube, the distal-side tube and the proximal-side tube are connected in such a way that a lumen of the distal-side tube and a lumen of the proximal-side tube communicate with each other, a guide wire port that opens to the outside is provided in the distal-side tube, and the inner diameter of the lumen of the proximal-side tube is smaller than the inner diameter of the lumen of the distal-side tube,

### CROSS REFERENCE TO RELATED APPLICATIONS

This application is a continuation application based on a PCT Patent Application No. PCT/JP2018/004207, filed on Feb. 7, 2018, whose priority is claimed on Japanese Patent Application No. 2017-025981, filed Feb. 15, 2017. The contents of both the PCT Application and the Japanese Application are incorporated herein by reference.

### BACKGROUND

### Technical Field

The present invention relates to a catheter for medical use, and more particularly, to a catheter tube used for a catheter for medical use through which a guide wire is inserted.

### Background Art

Conventionally, a catheter for medical use (hereinafter, may be simply referred to as a “catheter”) having a guide wire lumen through which a guide wire is introduced is known. In a general catheter placement procedure, first, the distal end of the guide wire is placed at an intended site in a patient's body via a forceps channel of an endoscope. Next, the proximal end of the guidewire protruding from the proximal end of the forceps channel is guided into a guide wire lumen of a catheter from the distal end of the catheter, then the catheter is advanced through the guidewire lumen (backload) to proceed along the guidewire. This allows the catheter distal end to be placed at the intended site.

In order to insert the catheter using the guide wire which distal end is fixed at the intended site, the length of an exposed part of the guide wire protruding from the proximal end of the forceps channel of the endoscope needs to be longer than the length of the guide wire lumen of the catheter, which length is defined as the length between the distal-side opening and the proximal-side opening of the guide wire lumen of the catheter. If the length of the protruding part of the guide wire coming out from the proximal end of the forceps channel is shorter than the length between the distal-side opening and the proximal-side opening of the guide wire lumen of the catheter, then it is difficult for an operator to keep the guide wire at the intended position during the insertion of the catheter. Therefore, it also becomes difficult to place the distal end of the catheter at the intended position.

When using a catheter in which the openings of the guide wire lumen are provided at the distal end portion and the proximal end portion of the catheter, the length of the guide wire extending from the forceps channel needs to be longer than the entire length of the catheter, and this makes the operation complicated. On the other hand, providing the proximal end opening (hereinafter referred to as “guide wire port”) of the guide wire lumen on the outer peripheral surface of the longitudinal middle portion of the catheter has been proposed and put to practical use.

In a catheter provided with a guide wire port on the outer peripheral surface of the longitudinal middle portion of the catheter, an axis of the proximal opening provided on the outer peripheral surface and that of the guide wire lumen make an angle with each other, and the extending directions are different each other. Therefore, the guide wire may not smoothly come out of the proximal opening.

In regard to this problem, in Published. Japanese Translation No. 2008-509726 of the PCT International Publication, it is described that a part of the outer peripheral surface is cut into a flap shape and pushed into a guide wire lumen, thereby forming a proximal-side opening and fanning an inclined surface that connects the guide wire lumen and the proximal-side opening. By guiding the guide wire along the inclined surface, the guide wire can be smoothly protruded from the proximal-side opening.

In the catheter described in Published. Japanese Translation No. 2008-509726 of the PCT International Publication, the proximal-side opening of the guide wire lumen is formed in a tube having two or more lumens.

Therefore, when the diameter of the catheter is reduced, the catheter tube becomes generally thin in order to secure the volume inside the lumen, and the tube rigidity and the kink resistance are reduced.

### SUMMARY

A catheter tube includes: a single-lumen distal-side tube; and a single-lumen proximal-side tube, wherein the distal-side tube and the proximal-side tube are connected in such a way that a lumen of the distal-side tube and a lumen of the proximal-side tube communicate with each other, a guide wire port that opens to an outside is provided on the distal-side tube, and an inner diameter of the single lumen of the proximal-side tube is smaller than an inner diameter of the single lumen of the distal-side tube.

In the catheter tube, a distal end of the proximal-side tube may have an inclined slope portion, and the guide wire port may be positioned to overlap at least a portion of the slope portion when viewed from a radial direction of the distal-side tube.

In the catheter tube, the lumen of the proximal-side tube may be formed at a position eccentric to a direction away from the guide wire port from a central axis of the proximal-side tube as viewed in a longitudinal direction.

According to each aspect of the present invention, the catheter tube can be suitably reduced in diameter, and the backloaded guide wire can be more smoothly protruded from the proximal-side opening.

### BRIEF DESCRIPTION OF THE DRAWINGS

FIG. 1 is a view showing the entire configuration of a catheter for medical use including a catheter tube according to a first embodiment of the present invention.

FIG. 2 is a cross-sectional view schematically showing the catheter for medical use.

FIG. 3 is a cross-sectional view schematically showing a modified example of the catheter for medical use.

FIG. 4 is a cross-sectional view schematically showing a catheter for medical use including a catheter tube according to a second embodiment of the present invention.

FIG. 5 is a cross-sectional view schematically showing a modified example of the catheter for medical use.

FIG. 6 is a cross-sectional view schematically showing a modified example of the catheter for medical use.

FIG. 7 is a cross-sectional view schematically showing a catheter for medical use including a catheter tube according to a third embodiment of the present invention.

### DETAILED DESCRIPTION OF THE PREFERRED EMBODIMENTS

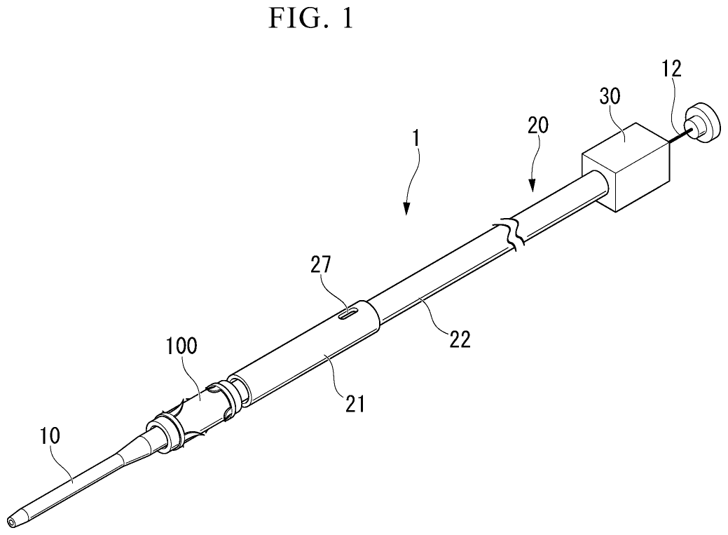

A first embodiment of the present invention will be described with reference to FIGS. 1 to 3.

FIG. 1 is a perspective view showing a state in which a stent 100 is attached to a catheter 1 for medical use into which a pusher tube 20 which is a catheter tube according to the present embodiment is incorporated. The catheter 1 includes an elongated guide catheter 10, a pusher tube 20 through which the guide catheter 10 is passed, and an operation unit 30 provided at a proximal end of the pusher tube 20.

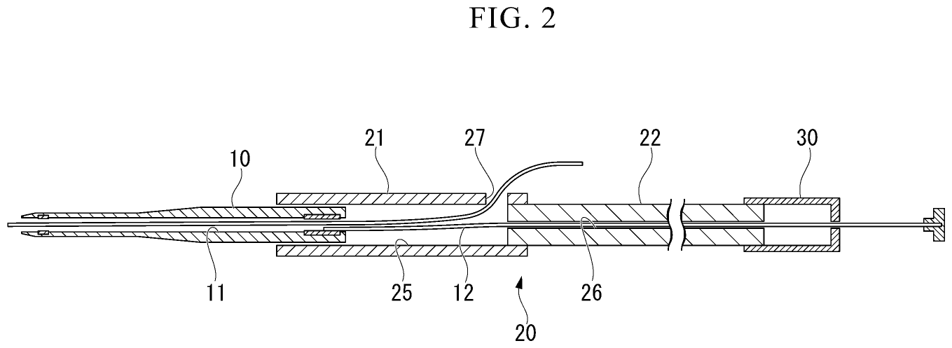

FIG. 2 is a cross-sectional view schematically showing the pusher tube 20 according to the present embodiment.

The guide catheter 10 is a tubular member formed of resin or the like, and includes a catheter lumen 11 having an inner diameter through which a guide wire can be passed. The catheter lumen 11 extends the entire length of the guide catheter 10 and is open at the distal end and the proximal end of the guide catheter 10. An operation wire 12 is connected to the proximal end of the guide catheter 10.

The pusher tube 20 is configured by connecting a first tube 21 (a distal-side tube) on the distal end side and a second tube 22 (a proximal-side tube) on the proximal end side. The first tube 21 is a tubular member having an inner diameter larger than the outer diameter of the guide catheter 10, and has a first lumen 25 into which the proximal end of the guide catheter 10 can enter. The second tube 22 has a second lumen 26 through which the operation wire 12 is passed. The first tube 21 and the second tube 22 are connected in such a manner that the first lumen 25 of the first tube 21 and the second lumen 26 of the second tube 22 communicate with each other.

A guide wire port 27 communicating with the first lumen 25 of the first tube 21 is formed on the outer peripheral surface of the first tube 21 at a position close to the joint between the first tube 21 and the second tube 22. Each of the first tube 21 and the second tube 22 is a single-lumen tube in which only one lumen is formed. The guide wire and the operation wire 12 are passed through the first lumen 25. Only the operation wire 12 is passed through the second lumen 26.

The operation unit 30 is formed in a substantially column shape, for example, using a resin or the like as a material. The operation wire 12 connected to the guide catheter 10 extends through the first and second lumens of the pusher tube 20 to the operation unit 30 and protrudes from the proximal end of the operation unit 30. The user can operate the pusher tube 20 advanced and retracted with respect to the guide catheter 10, by holding the proximal end portion of the operation wire 12 and holding the operation unit 30 and advancing and retracting the operation unit 30 with respect to the operation wire 12. The inner diameter of the pusher tube 20 is smaller than the outer diameter of the stent 100. Therefore, by advancing the pusher tube 20 with respect to the guide catheter 10, the stent 100 mounted on the guide catheter 10 can be pushed out and placed in the body.

The guide wire does not pass through the second lumen 26 of the second tube 22, and protrudes outward from a guide wire port 27 formed in the first tube 21. Therefore, it is not necessary to introduce a guide wire through the second tube 22. Thus, the inner diameter of the second lumen 26 of the second tube 22 can be smaller than the inner diameter of the lumen of the first tube 21.

If the second tube 22 was provided with the guide wire port 27, the second tube 22 would need two lumens. To make the diameter of the second tube 22 smaller, it would be necessary to make the second tube 22 thin in order to secure the volume inside the lumen, and the thin wall of the second tube would reduce the rigidity and kink resistance.

In the pusher tube 20 according to the present embodiment, only the second lumen 26 through which the operation wire 12 passes through the second tube 22 must be formed. Therefore, compared with the case where the guide wire port 27 is provided in the second tube 22, the pusher tube 20 according to the present embodiment can reduce the manufacturing cost. Even in the case of reducing the diameter of the pusher tube 20, particularly the second tube 22, it is easier to ensure enough thickness of the wall compared to the case where the guide wire port 27 is provided in the second tube 22, and the stiffness and kink resistance of the tube are less likely to decrease.

The guide wire is projected to the outside from a guide wire port 27 formed in the first tube 21 without passing through the connection portion between the first tube 21 and the second tube 22. Therefore, the guide wire is not hooked at the connecting portion when inserting and removing the guide wire.

Furthermore, in the second lumen 26 of the second tube 22 through which the operation wire 12 is passed, the clearance between the inner surface of the second lumen 26 and the operation wire 12 is smaller than diameter of the guide wire. Therefore, the backloaded guide wire does not accidentally enter the inside of the second lumen 26 of the second tube 22.

These allow the backloaded guide wire to project more smoothly from the guide wire port 27.

Next, the manufacturing method of the pusher tube 20 to make the pusher tube 20 comprised as mentioned above is described.

The second tube 22 is inserted on the proximal end side of the first tube 21 and the both parts are overlap-bonded by an adhesive. For example, the first tube has an outer diameter of 2.70 mm and an inner diameter of 2.20 mm, and the second tube has a diameter slightly smaller than the outer diameter of 2.20 mm and an inner diameter of 1.00 mm. The first tube 21 and the second tube 22 are connected in a state in which the second tube 22 is inserted into the first tube 21.

The connection between the first tube 21 and the second tube 22 is not limited to the above. In a state where the second tube 22 is inserted on the proximal end side of the first tube 21, the overlapping portion of the two tubes may be joined by heat fusion. In this case, the heat-shrinkable tubes may be disposed outside the overlapping portion of the two tubes, and the heat-shrinkable tubes may be heated to fuse the two tubes.

One tube may be connected without being inserted into the other tube. The ends of the first tube 21 and the second tube 22 may be connected by an adhesive or heat fusion.

After the first tube 21 and the second tube 22 are connected, a guide wire port 27 if formed that communicates with the lumen of the first tube 21 at a position near the joint between the first tube 21 and the second tube 22 on the outer peripheral surface of the first tube 21. A known technique is used to form the guide wire port 27.

As described above, according to the pusher tube 20 according to the present embodiment, the guide wire does not pass through the second lumen 26 of the second tube 22 and protrudes outward from the guide wire port 27 formed in the first tube 21. Therefore, it is not necessary to form a lumen for a guide wire in the second tube 22, which is effective in reducing the diameter of the second tube 22 and reducing the manufacturing cost.

The guide wire does not pass through the connection portion between the first tube 21 and the second tube 22. Therefore, the backloaded guide wire can be protruded from the guide wire port 27 more smoothly.

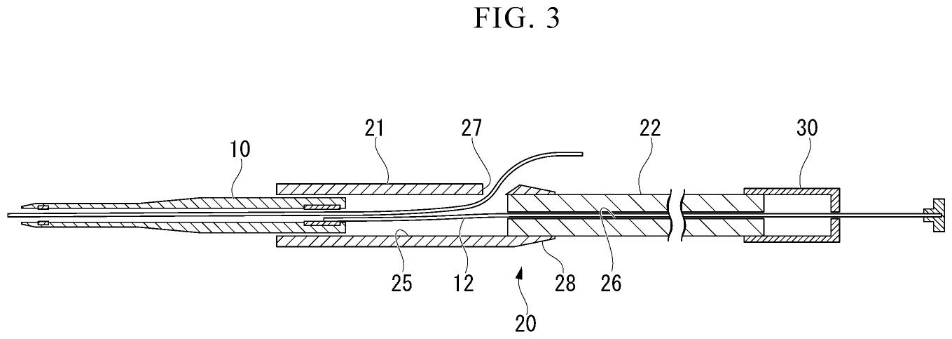

FIG. 3 is a cross-sectional view schematically showing a modified example of the pusher tube 20 according to the present embodiment.

As shown in FIG. 3, in the connection between the first tube 21 and the second tube 22, a reduced diameter portion 28 may be formed on the proximal end side of the outer peripheral surface of the first tube 21 such that the outer diameter decreases toward the proximal end side. This configuration reduces clog due to the thickness of the connection portion between the first tube 21 and the second tube 22 and improves the operability when inserting the catheter 1 for medical use into an endoscope.

For example, about 10 mm from the proximal end of the first tube, the outer diameter of the first tube 21 is reduced by about 0.05 to 0.2 mm.

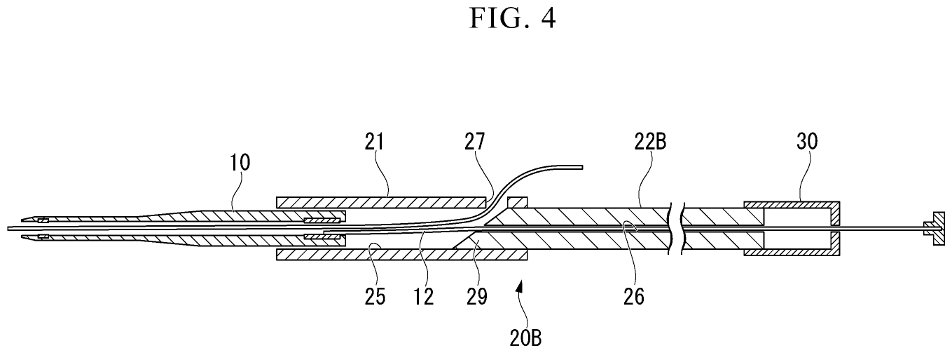

Next, a second embodiment of the present invention will be described with reference to FIGS. 4 to 6. The present embodiment differs from the first embodiment in that a slope portion 29 is provided on the distal end side of the second tube. In the following description, the same reference numerals are given to the same configuration and the like as those described above, and the redundant description will be omitted.

FIG. 4 is a cross-sectional view schematically showing a pusher tube 20B according to the present embodiment. A second tube 22B of the second embodiment has a slope portion 29 on the distal end side of the pusher tube 20B. The slope portion 29 guides the guide wire to the guide wire port 27.

On the distal end side of the slope portion 29, that is, the distal end side of the second tube 22B, a position farthest from the guide wire port 27 when viewed in the longitudinal direction of the second tube 22B protrudes in the longitudinal direction.

At an intermediate portion of the slope portion 29, a slope is formed from the position farthest from the guide wire port 27 toward the closest position. The shape of the inclined surface may be flat or curved.

The proximal end side of the slope portion 29 has the same outer diameter as the outer diameter of the second tube 22B. A position closest to the guide wire port 27 as viewed in the longitudinal direction of the second tube 22B on the proximal end side of the slope portion 29 is disposed near the proximal-side opening of the guide wire port 27. The guide wire port 27 is at a position overlapping at least a part of the slope portion 29 when viewed from the radial direction of the first tube 21.

With this configuration, the guide wire is guided by the slope portion 29 to the guide wire port 27 and is smoothly discharged from the guide wire port 27.

When manufacturing the pusher tube 20B, the slope portion 29 is formed at the distal end of the second tube 22B before connecting the first tube 21 and the second tube 22B. For example, the distal end of the second tube 22B is cut off obliquely. Thereafter, the first tube 21 and the second tube 22B are connected. Therefore, the formation of the slope portion 29 is easier than in a case when the slope portion is formed after the connection of the first tube 21 and the second tube 22B.

The connection aspect between the first tube 21 and the second tube 22 may be the same as in the first embodiment.

After connecting the first tube 21 and the second tube 22B, the guide wire port 27 is formed on the outer peripheral surface of the first tube 21 at a position close to the junction between the first tube 21 and the second tube 22B. More specifically, the guide wire port 27 is formed at a position overlapping at least a part of the slope portion 29 when viewed from the radial direction of the first tube 21. A known technique is used to form the guide wire port 27.

As described above, according to the pusher tube 20B according to the present embodiment, the guide wire does not pass through the second lumen 26 of the second tube 22B and protrudes outward from the guide wire port 27 formed in the first tube 21. Therefore, it is not necessary to form a lumen for a guide wire in the second tube 22B, which is effective in reducing the diameter of the second tube 22B and reducing the manufacturing cost.

The guide wire does not pass through the connection portion between the first tube 21 and the second tube 22B. The slope portion 29 can preferably guide the guide wire to the guide wire port and allow the backloaded guide wire to project more smoothly from the guide wire port 27.

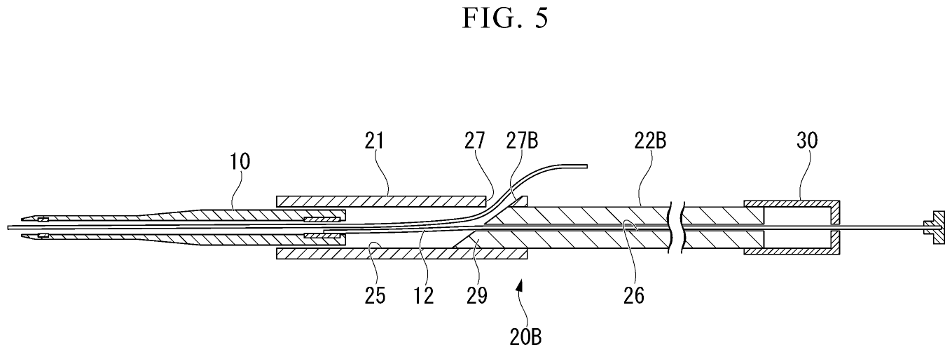

FIG. 5 is a cross-sectional view schematically showing a modification of the pusher tube 20B according to the present embodiment.

As shown in FIG. 5, an inclined portion 27B may be provided at the proximal-side opening of the guide wire port 27. A part of the proximal end side of the slope portion 29 can form a continuous surface with the inclined portion 27B of the proximal-side opening of the guide wire port 27. By providing the inclined portion 27B, the guide wire can be more suitably guided to the guide wire port, and the backloaded guide wire can be more smoothly protruded from the guide wire port 27.

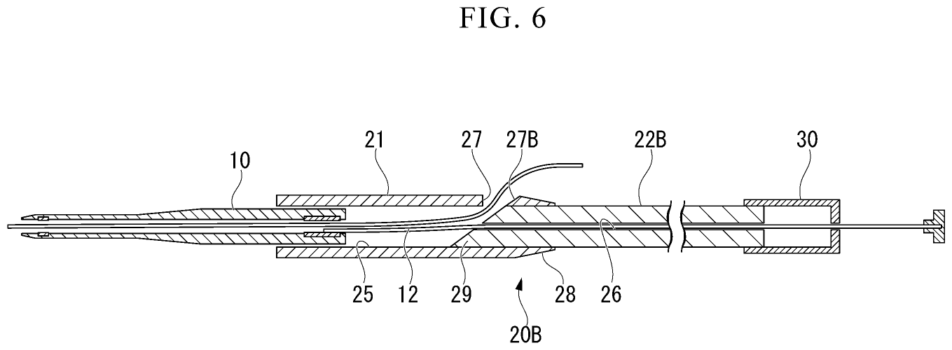

FIG. 6 is a cross-sectional view schematically showing a modified example of the pusher tube 20B according to the present embodiment.

As shown in FIG. 6, similarly to the first embodiment, the diameter-reduced portion 28 may be formed at the connection portion between the first tube 21 and the second tube 22B. The operability of the catheter 1 for medical use can be improved.

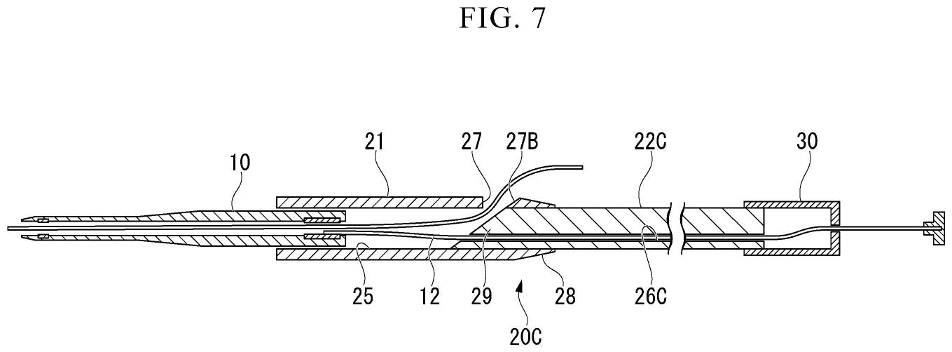

Next, a third embodiment of the present invention will be described with reference to FIG. 7. The present embodiment differs from the second embodiment in the position of the second lumen 26C of the second tube 22C. In the following description, the same reference numerals are given to the same configuration and the like as those described above, and the redundant description will be omitted.

FIG. 7 is a cross-sectional view schematically showing a pusher tube 20C according to the present embodiment. The second lumen 26C of the second tube 22C of the second embodiment is formed at a position eccentric to the direction away from the guide wire port 27 from the central axis of the second tube 22C when viewed along the longitudinal direction of the second tube 22C.

The operation wire 12 passes through the second lumen 26C located at a position far from the guide wire port 27 from the center of the second tube 22C as viewed in the longitudinal direction of the second tube 22C. Therefore, the guide wire is not easily influenced by the movement of the operation wire 12 and is easily guided by the guide wire port 27.

The second lumen 26C need not be in an eccentric position from the distal end side to the proximal end side of the second tube 22C. The same effect can be obtained even if only the opening of the second lumen 26C on the distal end side of the second tube 22C is in the eccentric position.

Similar to the first embodiment, the diameter-reduced portion 28 may be formed at the connecting portion between the first tube 21 and the second tube 22C. The operability of the catheter 1 for medical use can be improved.

Similar to the second embodiment, the slope portion 29 may be formed on the distal end side of the second tube 22C. The guide wire can be preferably guided to the guide wire port, and the backloaded guide wire can be protruded from the guide wire port 27 more smoothly.

As described above, according to the pusher tube 20C according to the present embodiment, the guide wire does not pass through the second lumen 26C of the second tube 22C and protrudes outward from the guide wire port 27 formed in the first tube 21. Therefore, it is not necessary to form a lumen for a guide wire in the second tube 22C, which is effective in reducing the diameter of the second tube 22 and reducing the manufacturing cost.

The guide wire does not pass through the connection portion between the first tube 21 and the second tube 22C. The slope portion 29 can preferably guide the guide wire to the guide wire port. This allows the backloaded guide wire to project more smoothly from the guide wire port 27.

The eccentric second lumen 26C of the second tube 22C can guide the guide wire to the guide wire port more suitably. This allows the backloaded guide wire to project more smoothly from the guide wire port 27.

The operability of the catheter 1 for medical use can be improved by forming the reduced diameter portion 28 at the connection portion between the first tube 21 and the second tube.

The present invention can be widely applied to a catheter tube, can preferably reduce the diameter of the catheter tube, and can smoothly project the backloaded guidewire from the proximal-side opening.

What is claimed is:

1. A catheter tube comprising:
a distal-side tube having a single lumen and provided on a distal side of the catheter; and
a proximal-side tube having a single lumen and provided on a proximal side of the catheter,
wherein the distal-side tube and the proximal-side tube are connected in such a way that the single lumen of the distal-side tube and the single lumen of the proximal-side tube communicate with each other,
a guide wire port that opens to an outside is provided on the distal-side tube, and
an inner diameter of the single lumen of the proximal-side tube is smaller than an inner diameter of the single lumen of the distal-side tube.

2. The catheter tube according to claim 1, wherein
the proximal-side tube has a slope portion having an inclined surface at the distal end thereof, and
the guide wire port is positioned to overlap at least a portion of the slope portion when viewed from a radial direction of the distal-side tube.

3. The catheter tube according to claim 1, wherein
the single lumen of the proximal-side tube is formed at a position eccentric to a direction away from the guide wire port from a central axis of the proximal-side tube as viewed along a longitudinal direction.
